(19) United States
(12) Patent Application Publication
(10) Pub. No.: US 2010/0173417 A1
(43) Pub. Date: Jul. 8, 2010

(54) DIAGNOSTIC METHOD

(76) Inventors: Lennart Minthon, Malmo (SE); Elisabet Londos, Malmo (SE)

Correspondence Address: MERCHANT & GOULD PC, P.O. BOX 2903, MINNEAPOLIS, MN, 55402-0903, US

(21) Appl. No.: 12/651,631

(22) Filed: Jan. 4, 2010

Related U.S. Application Data
(60) Provisional application No. 61/142,352, filed on Jan. 4, 2009.

(51) Int. Cl. G01N 33/20

(52) U.S. Cl. 436/79

(57) ABSTRACT

The invention relates to a method to determine the Mg²⁺ and/or Ca²⁺ levels in a cerebrospinal fluid obtained from a mammal, wherein Mg²⁺ and/or Ca²⁺ levels are connected to an α-synucleinopathy associated disease or disorder as well as a kit to be used in said method.

This application claims benefit of U.S. Ser. No. 61/142,352, filed 4 Jan. 2009 and which application is incorporated herein by reference. To the extent appropriate, a claim of priority is made to the above disclosed application.

### FIELD OF THE INVENTION

The invention relates to a method to determine the Mg²⁺ and/or Ca²⁺ levels in a cerebrospinal fluid obtained from a mammal, wherein Mg²⁺ and/or Ca²⁺ levels are connected to an α-synucleinopathy associated disease or disorder as well as a kit to be used in said method.

### BACKGROUND OF THE INVENTION

The neuropathogenic findings of for example dementia with Lewy bodies (DLB) show a wide range of anatomic range. Lewy bodies, mainly composed of α-synuclein in an aggregated state (Baba et al., 1998; Tamamizu-Kato et al., 2006), are found from the brain stem to the cortex. In addition, many cases there is concurrent Alzheimers disease (AD) pathology in the form of beta-amyloid (Aβ)-containing senile plaques. The invariable finding of Lewy bodies in definite DLB has put much focus on the involvement of α-synuclein in the pathogenesis of the disease. Together with other neurodegenerative diseases that are characterized by abnormal α-synuclein aggregation such as Parkinson's disease and multiple system atrophy, DLB have been designated a synucleinpathy (Weisman and McKeith, 2007). Similarly to the putative pathogenic role of Aβ in AD (Blennow et al., 2006), abnormal α synuclein oligomerization and aggregation have been proposed to be a primary cause of this group of disorders (Weisman and McKeith, 2007).

Several neurodegenerative diseases, including AD and Parkinson's, are characterized by modified copper and zinc homeostasis in the brain (Lovell et al., 1998) and in the cerebrospinal fluid (CSF) (Molina et al., 1998; Basun et al., 1991; Jimenez-Jimenez et al., 1998). These changes seem to contribute either directly or indirectly to increased oxidative stress, an important factor in neuronal toxicity (Morgan et al., 2004). Furthermore, copper and zinc are also involved in inflammatory reactions, e.g. in the synthesis and secretion of interleukin-2, which may play a role in neurodegeneration (Todorich and Connor, 2004). When coupled to misfolded β-amyloid Aβ, this modified metal homeostasis appears to be an important factor in the pathological progression of AD, a line of reasoning that may hold true also for the toxicity of α-synuclein (Uversky et al., 2002).

There are many examples for the detection/diagnosis of neurological disease by analysing samples obtained from a subject. Several of them involve the detection of biomarkers at a specific tissues or fluid of the body e.g. CSF. For example US 2007099203 describes a method for detecting presence or absence of a neurodegenerative disease e.g. Parkinson's disease or dementia with Lewy bodies, in a subject by detecting gene product expression in cerebrospinal fluid. Often the methods for determining or diagnosing a Lewy body associated disease involve multiple protein biomarkers (WO0002053, WO2004001421). As a consequence these methods are expensive and complicated to perform. As Lewy bodies basically are deposited misfolded α-synuclein, a number of diagnostic methods are aiming at detecting various forms of this protein (e.g. WO2007021255, WO9950300).

Of particular relevance in the perspective of this invention are methods that investigate different aspects of the cerebrospinal fluid by detecting or quantifying biomarkers, within this compartment. Analysis of Cerebrospinal fluid (CSF) has previously been performed for the diagnosis of dementia and neuroinflammatory diseases, but has been focused in detecting other types of biomarkers such as procalcitonin and α-synuclein (EP1904855 and EP1476758). WO2005001483 presents a number of protein biomarkers in CSF that can be used to distinguish between the two dementive disorders. Also genetic markers have been identified, that helps determining whether a subject is at risk of developing a neurological disease (US2005191685).

At present the diagnosis of a patient with suspected dementia is a long and rather complex procedure requiring highly qualified expertise in the field. At a first diagnostic step memory and cognitive impairment of the patient is investigated. If dementia according the Diagnostic and Statistical Manual of Mental Disorders, revision IV (DSM IV) is set, a diagnostic program needs be initiated to identify the type of dementia. Different types of dementia require different medicinal treatment. In addition patients with dementia with Lewy bodies are very sensitive for some neuroleptic medicaments. There is today a great need for a simple, safe and reliable test for detecting or diagnosing a neurological disease or disorder derived from or characterized by α-synucleinopathy. Also, in order to find effective drugs against α-synucleinopathy associated diseases a method for reading the effect of future drugs in subjects is seriously needed. The present invention aims to meet these fundamental requests.

### SUMMARY OF THE INVENTION

The invention relates to the finding that for a number of disorders or diseases related to ageing there is a need to determine the Mg²⁺ and/or Ca²⁺ levels in the cerebrospinal fluid obtained from a mammal to be able to make a proper diagnose of what the mammal is suffering from. Thereby it is possible to improve the treatment of the mammal, such as a human being.

The average age of the population is increasing which causes new problems for the health system. The invention relates to the important findings that mammals that suffering from an α-synucleinopathy associated disease or disorder have increased levels Mg²⁺ and/or Ca²⁺ levels in the cerebrospinal fluid, which may evolve in a number of diseases or disorders. Therefore it is of importance to determine the levels of the mentioned ions.

In an first aspect the invention relates to a method to determine the Mg²⁺ and/or Ca²⁺ levels in a cerebrospinal fluid obtained from a mammal, wherein Mg²⁺ and/or Ca²⁺ levels are connected to an α-synucleinopathy associated disease or disorder comprising the steps of:

providing a first sample of a cerebrospinal fluid obtained from a mammal, analysing the levels of Mg²⁺ and/or Ca²⁺ in said first sample, providing a second sample of a cerebrospinal fluid obtained from a mammal, analysing the levels of Mg²⁺ and/or Ca²⁺ in said second sample and comparing the levels of ions in said first and said second sample in order to identify differences of said ion levels in said first and said second sample.

Thereby it is possible to identify a mammal which has an increased production/accumulation of the Mg²⁺ and/or Ca²⁺ amounts, wherein the increased levels are connected to an α-synucleinopathy associated disease or disorder. Example on diseases as well as disorders that may be analysed includes Parkinson's disease, dementia with Lewy bodies, MSA or Hallervorden-Spatz syndrome. Specifically mammals, such as human beings which suffers from dementia with Lewy bodies.

In a second aspect the invention relates to a kit for the detection of Mg²⁺ and/or Ca²⁺ in cerebrospinal fluid connected to an α-synucleinopathy associated disease or disorder comprising a reagent for quantification of calcium and/or magnesium ions in the form of the dyes calmagite or phenolsulphonephthalein or chromatic interfering substances—vanadate ions and/or Chlorophosphanazo-III or reagents similar thereto and optionally tubes to store collected cerebrospinal fluid samples.

In a final aspect the invention relates to the use of the method as defined above or the kit as defined above to identify/develop pharmaceutical compositions against α-synucleinopathy associated diseases or disorders.

### BRIEF DESCRIPTION OF THE FIGURES

FIG. 1 shows the correlation between cerebrospinal fluid Mg and Ca levels among DLB patients (mg/L).

FIG. 2 shows receiver operating characteristic (ROC) curves of CSF-Mg, CSFCa and CSF-Cu for differentiation of DLB patients from all patients with Alzheimer's disease (ADVasc and AD). The areas under the ROC curve for the analyzed metals with 95% confidence interval were: CSF-Mg, 0.92 (0.86-0.97), CSF-Ca 0.84 (0.76-0.92) and CSF-Cu 0.66 (0.57-0.76), respectively.

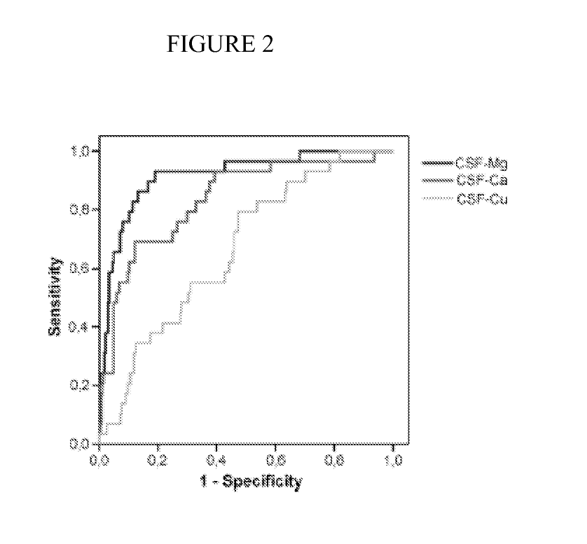

FIG. 3 shows the distribution of PTH and Cystatin C in each group of example 4.

### DETAILED DESCRIPTION OF THE INVENTION

### Definitions:

The uses of the word progression of the α-synucleinopaty disease herein relates to the disease state and also include the identification of how far the α-synucleinopathy associated disease has progressed and at what rate the disease progresses, as referring to the amount of Lewy bodies in the cerebral tissue in an individual.

The word mammal or subject herein is intended to include any mammal such as a human, dog, cat, soliped and cloven-hoofed animal or rodent such as a rabbit or a guinea pig or a murine such as a rat or a mouse.

The main diseases associated with the presence of Lewy bodies i.e. α-synucleinopathy is selected from the group consisting of Parkinson's disease and dementia with Lewy bodies as well as Hallervorden-Spatz syndrome and MSA.

A pharmaceutical composition against an α-synucleinopathy associated disease herein refers to a composition or drug, that removes Lewy bodies and/or Lewy neurites, repress or retards the development of Lewy bodies and/or Lewy neurites. The pharmaceutical composition may also decrease Mg²⁺ and/or Ca²⁺ levels in CSF or repress the further increase of Mg²⁺ and/or Ca²⁺ levels in the cerebrospinal fluid in an individual. The medication may also be a composition chelating Mg²⁺ and/or Ca²⁺ levels in the cerebrospinal fluid of an individual.

Lewy bodies herein refers to an eosinophilic cytoplasmic inclusion that consists of a dense core surrounded by a halo of 10-nm wide radiating fibrils, the primary structural component of which is α-synuclein.

Lewy bodies and Lewy neurites are mainly composed of aggregated α-synuclein proteins. The α-synucleinopathy associated disease may therefore be related to Lewy body and Lewy neurite associated disease.

### Invention

There is to the inventors' knowledge no prior study of metals in DLB patients as a representative group of α-synucleinopathy associated diseases or disorders in CSF or plasma. And in particular it is new to characterize the progression/development of α-synucleinopathy associated disease or disorder by quantifying Mg²⁺ and/or Ca²⁺ levels in CSF. The clinical material obtained from patients used in the study is superior to what has been available and used in other studies developing methods to characterize the progression/development of α-synucleinopathy associated diseases. For example, Ca, Mg, or Cu concentrations in CSF in did not show correlations with α-synucleinopathy associated disease (Jimenez-Jimenez et al., 1998; Forte et al., 2004). Most likely this was due to a different and less heterologos group of patients from which the material for analysis was obtained. Thus, the high quality of the patient material demonstrates the applicability of quantification of Ca-, Mg-, Cu-ions in CSF and relate to α-synucleinopathy associated diseases.

In the study performed by the inventors, metal concentration was measured in the CSF and plasma of DLB patients compared to AD patients, patients with AD and minor vascular components (AD-Vasc) and healthy volunteers. Interestingly, the inventors identified that the concentration of the Mg²⁺ and/or Ca²⁺ levels in the CSF was increased in the human beings that was not identified as being healthy volunteers.

By the experimental data obtained during the study performed by the inventors it is for the first time possible to determine the Mg²⁺ and/or Ca²⁺ levels in a cerebrospinal fluid, wherein said metal levels are connected to α-synucleinopathy associated disease or disorder obtained from a mammal comprising the steps of: providing a first sample of a cerebrospinal fluid obtained from a mammal, analysing the levels of Mg²⁻ and/or Ca²⁺ in said first sample, providing a second sample of a cerebrospinal fluid obtained from a mammal, analysing the levels of Mg²⁺ and/or Ca²⁺ in said second sample and comparing the levels of ions in said first and said second sample in order to identify differences of said ion levels in said first and said second sample. The first and said second samples are obtained from the same or different mammals. For example said first sample may be obtained from a mammal suffering from an α-synucleinopathy associated disease or disorder and said second sample is obtained from a healthy mammal or from a population of healthy mammals not suffering from an α-synucleinopathy associated disease or disorder. Examples of mammals are identified herein and may be selected from the group consisting of human beings, mouse, rats, guinea pigs, cats, dogs or genetically versions thereof Both Magnesium and Calcium belongs to second group (“alkaline earth metal”) of the periodic table, period 3 and 4 respectively. Thus, both are categorised in a period less than 5 in said table.

In one embodiment the mammal is a human being. In that particular embodiment the human being suffering from an α-synucleinopathy associated disease or disorder should have a Mg²⁻ level of at least 27.3 mg/L and/or a Ca²⁺ level of at least 48.0 mg/L, such as a Mg²⁺ level of at least 27.5, 28.0, 28.5, 29.0 or 29.3 mg/L and/or a Ca²⁺ level of at least 48.5, 49.0, 49.5, 50.0 or 50.7 mg/L. One example being a human being suffering from an α-synucleinopathy associated disease or disorder should have a Mg²⁺ level of at least 29.3 mg/L and a Ca²⁺ level of at least 50.7 mg/L. The method is also applicable to distinguish between dementia of Lewybody type and dementia of the Alzheimer type.

The invented method may be used to determine the progression of an α-synucleinopathy associated disease or disorder, for diagnosing an α-synucleinopathy associated disease or disorder, for tuning the dosage of a medicament against an α-synucleinopathy associated disease or disorder or for screening for pharmaceutically active compounds against α-synucleinopathy associated diseases or disorders, such as a disease or disorder is selected from the group consisting of Parkinson's disease, dementia with Lewy bodies, MSA or Hallervorden-Spatz syndrome. One example is dementia with Lewy bodies.

More specifically the method includes diagnosing a patient for having or starting to develop an α-synucleinopathy related disease such as Parkinson's disease, dementia with Lewy bodies, Multiple system atrophy (MSA) or Hallervorden-Spatz syndrome. By analysing a sample of CSF, obtained from a patient, for the concentration levels of Mg²⁺ and/or Ca²⁺ and compare these values with samples of CSF obtained from individuals not suffering from an α-synucleinopathy associated disease. When the concentration of Mg²⁺ and/or Ca²⁺ in CSF obtained from the individual to be diagnosed is high, relatively individuals not having an α-synucleinopathy associated disease, the individual diagnosed is indicated to have an α-synucleinopathy associated disease. The invention is particularly useful to identify patients with an α-synucleinopathy associated dementia within patient groups showing dementia according the Diagnostic and Statistical Manual of Mental Disorders, revision IV (DSM IV).

The invention further includes a method to tune/adjust the dosage of a medicament against α-synucleinopathy associated disease that is suitable for an individual going through a medicinal treatment against an α-synucleinopathy associated disease. Patients' differ in individual physiology and individual genotypes which may make a standardized dose of a medication not be optimal for treating a patient with an α-synucleinopathy associated disease. The present invention makes it possible to individually adjust the dosage of a medicament to an individual undergoing a medicinal treatment against an α-synucleinopathy associated disease. The method includes analysing a sample for the levels of Mg²⁺ and/or Ca²⁺ in a sample of cerebrospinal fluid obtained from a patient before the administration of a dosage of the medicament against α-synucleinopathy associated disease. Then said levels of ions are determined in another sample of cerebrospinal fluid obtained from the same patient at a time point during and/or after the administration of a dosage of the medicament against α-synucleinopathy associated disease. The method may also include that both the samples analysed have been obtained from a patient at different time points after the administration of a medicament against an α-synucleinopathy associated disease has commenced. The method may also include that several samples of CSF, obtained from the patient at different time points undergoing a medicinal treatment, are analysed for the concentration of Mg²⁺ and/or Ca²⁺. The level of said ions in the other sample relative the first sample is an indication of the effect of the dosage of the medicament against an α-synucleinopathy associated disease.

The invention further includes a method of screening/testing for pharmaceutical compounds active against the development of α-synucleinopathy associated diseases. The method comprising, determining the concentration of Mg²⁻ and/or Ca²⁺ in a first sample of CSF obtained from a mammal diagnosed with or have an α-synucleinopathy associated disease before the individual has been exposed to a compound undergoing a drug screening/testing. A second CSF-sample obtained from the same individual, but after the exposure of the compound undergoing a drug screening/testing are analysed for the concentration level of Mg²⁺ and/or Ca²⁺. The determined ion concentrations in the samples are compared. When an altered level of Mg²⁺ and/or Ca²⁻ in the CSF sample is detected in a sample obtained from an individual, after that a specific compound has been administered relative the first sample, a pharmaceutical effect related to an α-synucleinopathy associated disease are indicated.

The animal may for example be a human, dog, cat, ape or a rodent such as a rabbit, guinea pig, or murine such as a mouse or rat. In principle said animal may be of any kind from which it is possible to obtain a sample of CSF. The animal may be an individual belonging to the group of animal disease models for α-synucleinopathy associated diseases, drug-induced parkinsonism included. The animal may also be a genetic knock-out suitable for investigating α-synucleinopathy associated diseases such as animals being knocked out in 26S proteasomal depletion, Psmc1 or Nurr1 or parkin knock-out or any other genetic models that are suitable for investigating α-synucleinopathy associated disease.

The invented method also includes determining the progression of an α-synucleinopathy associated disease such as Parkinson's disease, dementia with Lewy bodies, Multiple system atrophy (MSA) or Hallervorden-Spatz syndrome. By analysing a sample of CNF obtained from a patient for the concentrations of Mg²⁺ and/or Ca²⁺ and compares these corresponding values with statistical curve derived from samples obtained from patients that have developed an α-synucleinopathy related disease. The samples for the statistical curve may have been obtained from the patients that have developed an α-synucleinopathy related disease at different (early and late) stages of disease development. Thereby a curve of the concentration of Mg²⁺ and/or Ca²⁺ in CSF relative α-synucleinopathy related disease development can be made. Samples obtained from individuals that do not have α-synucleinopathy related diseases are used as reference. The concentration determined from the sample obtained from a patient with α-synucleinopathy associated diseases give information about how far the disease has developed and/or to what extent Lewy bodies have started to form in the neuro-tissue. This is useful in order to determine and adjust the pharmaceutical and therapeutic treatment suitable for the patient.

The method also includes determining the rate of the α-synucleinopathy formation in a patient at a certain time. First, Mg²⁻ and/or Ca²⁺ concentrations in a sample of CSF obtained from a patient are determined The determined concentration levels are related to corresponding ion concentration levels obtained from individuals not suffering from an α-synucleinopathy associated disease. When said ion levels are markedly higher in the sample obtained from the patient compared to the concentration in samples obtained healthy individuals, the rate of α-synucleinopathy formation is high. Furthermore, when the concentration levels are only slightly higher in samples obtained from patients relative samples obtained from healthy individuals.

Within the scope of the invention is also to use any of the above specified methods to develop new drugs or further develop existing drugs against α-synucleinopathy associated disease. The use of the method of the invention includes the development of any type of drug that has a pharmaceutical activity against α-synucleinopathy associated diseases. The drugs may for example be of Mg²⁺ and/or Ca²⁺ chelating type of compounds, such as of type tetracycline family chelators, EDTA, EGTA, Sparteine (the predominant alkaloid in Lupinus mutabilis) or another pharmaceutical acceptable compound capable of binding/chelate Mg²⁺ and/or Ca²⁺.

The drugs may also be Mg²⁺ and/or Ca²⁺ homeostasis regulators, i.e. compounds that regulate the levels of Mg²⁺ and/or Ca²⁺ in subcellular organelles, including mitochondria, versus outside organelle or that regulate the levels of Mg²⁺ and/or Ca²⁺ in a cell versus outside cell. Examples of classes of calcium channel regulators are Dihydropyridine, Phenylalkylamine, Benzothiazepine or other drugs with similar uses. Dihydropyridine calcium channel blockers may for example be Amlodipine (Norvasc), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Efonidipine (Landel), Felodipine (Plendil), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas) or derivatives or salts or esters thereof Phenylalkylamine calcium channel blockers may for example be Verapamil (Calan, Isoptin), Gallopamil (Procorum, D600) or derivatives or salts or esters thereof Benzothiazepine calcium channel blockers may for example be Diltiazem (Cardizem) or derivatives or salts or esters thereof Other classes of pharmaceutical agents that have overlapping effects as calcium channel blockers include ACE inhibitors, beta-blockers, and nitrates. Other drugs where the present method can be used, may for example counter act to impaired electron transport chain (ETC) functioning such as antihistamine drugs that influences divalent ion (e.g. calcium and magnesium) permeability of the membranes of mitochondria e.g. Dimebolin. Other compounds that also can be of interest in the treatment of α-synuclinopathy associated diseases are gamma-carbolines, including the neuroleptic agents flutroline, gevotroline hydrochloride (WY-47384) and carbidine, and another antihistamine mebhydrolin (mebhydroline).

The invention also includes testing the effect of each of the above listed drugs or any possible combination thereof (inter- and intra-drug-groups). Especially acetyl cholinesterase inhibitors have potential to be used as one of the ingredients in combinatorial treatments against α-synucleinopathy associated diseases.

Neuroprotective treatments are at the forefront of PD research, but are still under clinical scrutiny (Bonuccelli and Del Dotto, 2006). These agents could protect neurons from cell death induced by disease presence resulting in a slower progression of disease. Agents currently under investigation as neuroprotective agents include anti-apoptotic drugs (CEP 1347 and CTCT346), lazaroids, bioenergetics, antiglutamatergic agents and dopamine receptors (Djaldetti and Melamed, 2002). Clinically evaluated neuroprotective agents are the monoamine oxidase B inhibitors selegiline (The Parkinson Study Group, (N Engl J Med.), 1993) and rasagiline, dopamine agonists, and the complex I mitochondrial fortifier coenzyme Q10. Alpha6beta2 nAChR may represent a relevant target for PD therapeutics (Quik et al., 2007). The invented method described herein includes measuring the effects also of any of these neuroprotective agents.

In a further embodiment the invention relates to a kit for the detection of Mg²⁺ and/or Ca²⁺ in cerebrospinal fluid comprising a reagent for quantification of calcium and/or magnesium ions in the form of the dyes calmagite or phenolsulphonephthalein or chromatic interfering substances—vanadate ions and/or Chlorophosphanazo-III or reagents similar thereto and optionally tubes to store collected cerebrospinal fluid samples. The kit may be used in the method defined above. Several methods are available for quantifying Ca- and/or Mg-ions in bodyfluids. There are standard Magnesium and Calcium quantification kits available on the market based on e.g. calmagite dye for detecting magnesium and phenolsulphonephthalein dye for detecting calcium. EP1918717 discloses chromatic interfering substances in which the sample is analysed photometrically. Reagents for determining Calcium in body fluids comprising Chlorophosphanazo-III, an anionic surfactant and at least on kind of a component selected from the group consisting of amines (JP2008058033) and Chlorophosphanazo-III in combination with vanadate (EP16115029) are new possibilities available.

In a final embodiment the invention relates to the use of the method as defined above as well as the kit as defined above to identify/develop pharmaceutical compositions against α-synucleinopathy associated diseases or disorders, wherein said α-synucleinopathy associated disease or disorder is selected from the group consisting of Parkinson's disease, dementia with Lewy bodies, MSA or Hallervorden-Spatz syndrome, such as dementia with Lewy bodies.

The invention also relates to the use of the above identified method and kit to make a diagnose of a mammal suffering from dementia such as Parkinson's disease, dementia with Lewy bodies, MSA or Hallervorden-Spatz syndrome, such as dementia with Lewy bodies.

### Examples

### Example 1A

### Definition of Patient Groups and Sample Collection

CSF and plasma samples were obtained from 29 patients with DLB, 174 with AD and 90 with AD-Vase. All patients who had completed a full investigation of medical history, cognitive test (MMSE), computerized tomography of the brain, measurement of relative cerebral blood flow, blood, and CSF analyses, were selected consecutively from patients attending the Neuropsychiatry Clinic, University Hospital MAS, Malmo, Sweden between 1999 and 2003. The patients fulfilled the clinical criteria for AD (NINCDSADRDA) (McKhann et al., 1984) or the consensus criteria for DLB (McKeith et al., 1996, 2005). No patient fulfilled the NINCDS-AIREN or DSM IV criteria for vascular dementia. AD-Vasc was defined as fulfillment of the clinical criteria for AD and a history with at least one suspected cerebrovascular insult and/or minor ischemic insult on computerized tomography without any clear causative effect on the development of clinical dementia. In addition MMSE score, CSF and plasma was gathered from 51 healthy volunteers. EDTA was used as anticogulant for the plasma collection. All plasma and CSF samples were stored at −80° C. until analysis.

Between-group comparisons of demographic data

| | | | | | Significant |
|---|---|---|---|---|---|
| | | | | | differences |
| | | | | | P < 0.05 |
| | DLB | AD | AD-Vasc | Controls | (DLB vs other) |
| N | 29 | 174 | 90 | 49 | |
| Gender male/female (n) | 17/12 | 52/122 | 33/57 | 15/34 | DLB vs Controls, |
| | | | | | AD |
| Age¹ | 74 (54-84) | 74 (52-86) | 77 (57-87) | 73 (60-87) | No |
| Disease duration (yrs)^{1a} | 2 (0-8) | 2 (0-10) | 2 (0-8) | Na | No |
| MMSE (score 0-30)^{1a} | 23 (14-29) | 22 (2-30) | 22 (6-30) | 30 (27-30) | DLB vs Controls |
| CSF-Albumin (g/L)^{2a} | 0.29 +− 0.16 | 0.27 +− 0.11 | 0.28 +− 0.12 | 0.25 +− 0.08 | No |
| S-Albumin (g/L)^{2a} | 35.5 +− 3.11 | 36.4 +− 3.61 | 35.9 +− 3.70 | 36.0 +− 2.45 | No |
| Q-Alb^{2a} (×10⁻³) | 8.2 +− 3.2 | 7.3 +− 2.4 | 7.8 +− 3.6 | 7.4 +− 3.2 | No |

Dementia with Lewy bodies (DLB),
Alzheimer's disease (AD),
Alzheimer's disease with minor vascular components (AD-vasc),
number (N),
not applicable (na)
mini mental state examination (MMSE),
in cerebrospinal fluid (CSF-),
in serum (S-)
¹median (range)
²mean (S.D.)
^{a}DLB n = 26-28, AD n = 166-174, AD vasc = 83-90, controls n = 48-49.

### Example 1B

Sample Analysis

The total concentrations in CSF and plasma of magnesium (Mg), calcium (Ca), manganese (Mn), iron (Fe), copper (Cu), zinc (Zn), rubidium (Rb), strontium (Sr), and cesium (Cs) were determined by inductively coupled plasma-mass spectrometry (ICP-MS; Thermo X7, Thermo Elemental, Winsford, UK) in accordance with Gerhardsson and colleagues (submitted for publication). To ensure the accuracy of the analytical methods and results, samples with 2% HNO3 and quality control (QC) samples were analyzed along with the collected samples (Seronorm Trace Elements Serum Lot MI0181; SERO AS, Billingstad). The background contamination of the collection vessels was below the detection level of the analytical method used, and the obtained values for the QC samples showed good agreement with the recommended concentrations. Albumin levels in CSF and serum were measured by immunonephelometry on a Beckman Immage Immunochemistry system (Beckman Instruments). The albumin ratio (Q(alb)) was calculated as CSF albumin/serum albumin.

### Example 1C

### Statistical Analysis of Data

SPSS 14.0 was used for statistical analysis. Since the metal concentrations were not normally distributed a nonparametric Kruskal-Wallis one-way analysis of variance was performed followed by Mann-WhitneyU-test for continuous variables to analyze differences between DLB and controls, AD patients and AD-Vasc patients. Adjustment for multiple comparisons was performed using the Bonferroni method. Spearman's rank order correlation was performed post hoc to analyze the relationship between the two metals that were significantly increased in the DLB patients compared to the controls. The Youden method (Youden, 1950) was used to establish the optimal cut-off point to assess the diagnostic accuracy of the different metal concentrations in distinguishing DLB from AD.

### Example 2

### Concentrations of Mg2+ and Ca2+ in CSF and Plasma.

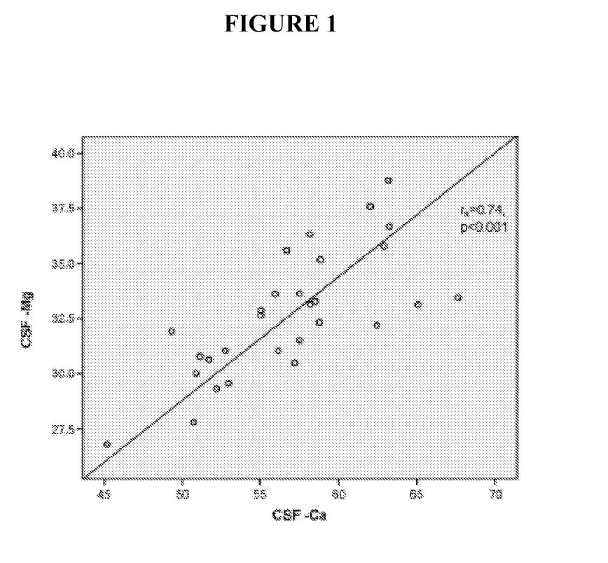

Mg, Ca and Cu differed in concentration in CSF samples from DLB patients compared to the other groups (Tables 2 and 3 and FIG. 1), however, after correction for multiple comparisons differences in CSF Cu concentrations failed to reach significance. Yet, DLB patients displayed higher Ca and Mg levels in CSF when compared to all other groups in a statistically robust manner (p<0.001). Furthermore, CSF-Mg and CSF-Ca correlated strongly within the DLB group (rs=0.74, p<0.001) (FIG. 1). In addition, Cs in CSF was elevated among the DLB patients compared to the AD patients (p<0.001) but not compared to the controls or the AD-Vasc patients. Rb and Cs CSF levels were borderline significantly increased in DLB compared to AD and ADVasc, respectively. Mn, Fe, Cu, Zn, Rb and Sr concentrations in CSF did not differ between DLB patients and the other groups (Table 2)

Patients with DLB had significantly increased Mg levels in their plasma compared to controls and AD patients (p<0.001), as well as compared to AD-Vasc patients (p<0.05), and showed increased Ca in their plasma compared to AD-Vasc patients (p<0.05). In addition, P—Cu was elevated among the DLB patients compared to patients with AD and AD-Vasc (p<0.05) but not compared to controls (Table 3) All DLB patients had P—Mg and P—Ca values within the normal reference range used at the local laboratory for clinical practice (Mg 0.7-1.2 mM; Ca 2.2-2.6 mM)

TABLE 2
Metal concentration in CSF for the different patient groups

| | | | | | DLB vs | DLB vs | DLB vs AD- |
|---|---|---|---|---|---|---|---|
| | DLB | Controls | AD-Vasc | AD | Controls p | AD p | Vasc p |
| Mg | 32.6 | 28.0 | 27.3 | 27.5 | 1.9 × 10⁻⁸*** | 1.8 × 10⁻¹²*** | 3.2 × 10⁻¹²*** |
| (mg/L) | (26.8-38.8) | (25.2-37.0) | (23.5-34.0) | (23.4-35.5) | | | |
| Ca | 56.2 | 49.6 | 49.8 | 49.9 | 1.5 × 10⁻⁶*** | 3.6 × 10⁻⁹ *** | 1.9 × 10⁻⁷ *** |
| (mg/L) | (45.2-67.6) | (44.0-67.7) | (42.8-64.4) | (41.5-67.2) | | | |
| Mn | 0.67 | 0.72 | 0.63 | 0.61 | 0.38 | 0.25 | 0.57 |
| (μg/L) | (0.37-2.43) | (0.41-2.02) | (0.21-2.04) | (0.25-3.36) | | | |
| Fe | 224 | 235 | 174 | 192 | 0.52 | 0.36 | 0.093 |
| (μg/L) | (148-269) | (122-403) | (106-438) | (100-434) | | | |
| Cu | 20.7 | 18.2 | 18.4 | 17.8 | 0.006* | 0.0036* | 0.010* |
| (μg/L) | (14.0-140.2) | (12.9-34.7) | (8.41-83.71) | (9.0-109) | | | |
| Zn | 21.0 | 17.0 | 17.0 | 17.6 | 0.39 | 0.10 | 0.17 |
| (μg/L) | (8.1-146) | (9-1033) | (8.41-66.6) | (9.0-138) | | | |
| Rb | 76.5 | 72.5 | 67.5 | 66.9 | 0.94 | 0.012 | 0.054 |
| (μg/L) | (55.5-155) | (51.2-115.5) | (46.0-118.1) | (38.1-121.8) | | | |
| Sr | 15.9 | 13.8 | 14.4 | 13.5 | 0.15 | 0.10 | 0.11 |
| (μg/L) | (8.5-34.6) | (5.1-33.1) | (6.4-83.8) | (5.7-65.4) | | | |
| Cs | 0.26 | 0.28 | 0.22 | 0.19 | 0.30 | 0.0002** | 0.011 |
| (μg/L) | (0.15-0.47) | (0.14-0.62) | (0.11-0.43) | (0.07-0.62) | | | |

All metal concentrations are presented as median (range).
Dementia with Lewy bodies (DLB),
Alheimer's disease (AD),
Alzheimer's disease with minor vascular components (AD-Vasc).
*<0.01;
**<0.001;
***<0.0001.

This study included more male than female DLB patients, a trend seen in most studies of DLB. However, there were no significant differences between genders regarding metal concentration in CSF or plasma in any of the studied groups.

TABLE 3
Metal concentration in plasma for the different patient groups

| | | | | | DLB vs | DLB vs | DLB vs |
|---|---|---|---|---|---|---|---|
| | DLB | Controls | AD-Vasc | AD | Controls p | AD p | AD-Vasc p |
| Mg | 22.8 | 21.1 | 21.4 | 21.3 | 0.00033** | 0.00024** | 0.0017* |
| (mg/L) | (18.5-27.6) | (18.0-27.5) | (13.4-25.3) | (15.4-28.7) | | | |
| Ca | 102.7 | 98.3 | 98.0 | 97.4 | 0.003* | 0.0017* | 0.00044** |
| (mg/L) | (87.6-123.2) | (82.5-142.1) | (71.3-113.7) | (83.0-125.5) | | | |
| Mn | 0.99 | 0.94 | 1.40 | 1.45 | 0.66 | 0.079 | 0.098 |
| (μg/L) | (0.61-2.50) | (0.63-2.24) | (0.51-4.49) | (0.48-5.95) | | | |
| Fe | 1.53 | 1.67 | 1.54 | 1.61 | 0.006* | 0.10 | 0.21 |
| (mg/L) | (0.99-1.88) | (1.05-3.54) | (0.75-2.96) | (0.72-3.24) | | | |
| Cu | 1.36 | 1.26 | 1.22 | 1.23 | 0.038 | 0.0013* | 0.0015* |
| (mg/L) | (0.98-2.25) | (0.81-3.43) | (0.78-2.04) | (0.79-2.23) | | | |
| Zn | 771 | 846 | 854 | 872 | 0.013* | 0.0027* | 0.0038* |
| (μg/L) | (605-1348) | (535-1371) | (555-1197) | (573-2500) | | | |
| Rb | 299 | 300 | 302 | 294 | 0.83 | 0.40 | 0.69 |
| (μg/L) | (225-455) | (225-433) | (186-438) | (171-481) | | | |
| Sr | 31.5 | 31.6 | 30.4 | 31.2 | 0.77 | 0.10 | 0.11 |
| (μg/L) | (19.5-79.9) | (13.1-62.7) | (14.6-176) | (12.0-166) | | | |
| Cs | 0.83 | 1.06 | 1.40 | 0.78 | 0.18 | 0.31 | 0.17 |
| (μg/L) | (0.50-9.69) | (0.55-3.71) | (0.35-9.03) | (0.36-9.94) | | | |

All metal concentrations are presented as median (range).
Dementia with Lewy bodies (DLB),
Alzheimer's disease (AD),
Alzheimer's disease (AD),
Alzheimer's disease with minor vascular components (AD-Vasc).
*<0.01;
**<0.001.

### Example 3

### Diagnostic Correlations

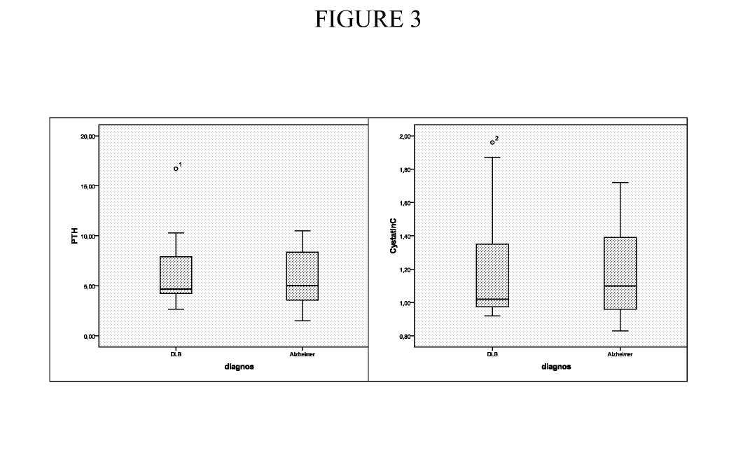

There was no significant correlation between CSF and plasma levels of Mg or Ca in the DLB group (Ca: rs=−0.20 p=0.30; Mg: rs=−0.25 p =0.20) or among the controls (Ca: rs=−0.23 p=0.10; Mg: rs=−0.27 p=0.55). Furthermore, Q(alb) did not correlate significantly with CSF-Ca or CSF-Mg in the DLB group (Ca: rs=0.31 p=0.13; Mg: rs=−1.45 p=0.48) or among the controls (Ca: rs=0.39 p=0.13; Mg: rs=−0-17 p=0.90). The CSF-Mg concentration could distinguish DLB patients from AD patients (AD and AD-Vasc) with a sensitivity of 93% and a specificity of 81%, when an optimal cut-off value of 29.3 mg/L was used. Moreover, the CSF-Ca concentration resulted in a sensitivity of 93% and a specificity of 63% for detection of DLB among the demented patients using the optimal cut-off value 50.7 (FIG. 3). A combination of the two variables yielded a sensitivity of 93% and a specificity of 85% when a positive result was defined as a combination of CSF-Mg≦27.3 mg/L and CSF-Ca≦48.0 mg/L. The plasma levels of the analyzed metals were unable to discriminate DLB from AD with small areas under the receiver operating characteristic curve (≦0.71).

### Example 4

11 patients with DLB and 22 AD patients, matched according to age and gender, were selected from a larger population of 29 DLB and 264 AD patients that participated in an earlier study described in the examples above. All patients had completed a full investigation of medical history, cognitive test (MMSE), computerized tomography of the brain, measurement of relative cerebral blood flow, blood, and CSF analyses and plasma trace element analysis. The patients fulfilled the revised consensus criteria for DLB or the clinical criteria for AD (NINCDS-ADRDA)

Plasma from all study subjects was analyzed in a single batch regarding levels of PTH (Immulite® 2000 Intact PTH, DPC-Diagnostic Products Corporation) and Cystatin C. EDTA was used as anticoagulant for the plasma collection. All plasma and CSF samples were stored at −73° C. until analysis.

### Statistical Analysis

SPSS 17.0 was used for statistical analysis. To avoid possible bias of non normal distribution, Mann Whitney U-test for continuous variables was to analyze differences between DLB and AD regarding age, CSF Ca and Mg, PTH and Cystatin C.

### Results

Demographics and analyzed data is presented in table 1. P-PTH and P-CystatinC in DLB and AD is presented in a boxplot in FIG. 1. CSF Mg and Ca was robustly increased in DLB compared to AD. There were no differences regarding P-PTH and P-Cystatin C between the groups.

### Conclusion

There were no differences between the DLB and AD group regarding P-PTH, a value representing parathyroidfunction, or Cystatin C, a value representing kidney function. Thus, the differences seen in CSF Mg and Ca are probably not explained by these possible confounding factors.

TABLE 1
Between-group comparisons of demographic
data and analyzed variables

| | | | Significant |
|---|---|---|---|
| | | | differences |
| | DLB | AD | P < 0.05 |
| N | 11 | 22 | Na |
| Gender | 6/5 | 11/11 | Na |
| male/female | | | |
| Age | 75 (66-83) | 75 (65-83) | 1 |
| CSF-Ca | 57.5 (45.2-63.2) | 49.9 (43.5-59.2) | 0.001 |
| CSF-Mg | 32.7 (26.8-38.8) | 27.1 (25.4-30.7) | <0.001 |
| P-CystatinC | 1.02 (0.92-1.96) | 1.10 (0.83-1.72) | 0.89 |
| P-PTH | 4.68 (2.66-16.70) | 5.04 (1.51-10.50) | 0.88 |

All variables are presented as median (range)

### LIST OF NON-PATENT LITERATURE REFERENCES

Bonuccelli U, Del Dotto P (2006). “New pharmacologic horizons in the treatment of Parkinson disease”. Neurology 67 (2): 30-38.
Baba M., Nakajo, S., Tu, P. H., Tomita, T., Nakaya, K., Lee, V. M., Trojanowski, J. Q., Iwatsubo, T., 1998. Aggregation of α-synuclein in Lewy bodies of sporadic Parkinson's disease and dementia with Lewy bodies. Am J Pathol. 152(4),879-84.
Basun H., Forssell, L. G., Wetterberg, L., Winblad, B., 1991. Metals and trace elements in plasma and cerebrospinal fluid in normal aging and Alzheimer's disease. J Neural Transm Park Dis Dement Sect. 3(4),231-58.
Blennow K., de Leon, M.J., Zetterberg, H., 2006. Alzheimer's disease. Lancet. 368(9533),387-403.
Djaldetti R, Melamed E (2002). “New drugs in the future treatment of Parkinson's disease”. J. Neurol. 249 Suppl 2: 1130-5. doi:10.1007/s00415-002-1206-2. PMID 12375061.
Forte G., Bocca, B., Senofonte, O., Petrucci, F., Brusa, L., Stanzione, P., Zannino, S., Violante, N., Alimonti, A., Sancesario, G., 2004. Trace and major elements in whole
Jimenez-Jimenez F. J., Molina, J. A., Aguilar, M. V., Meseguer, I., Mateos-Vega, C. J., Gonzalez-Munoz, M. J., de Bustos, F., Martinez-Salio, A., Orti-Pareja, M., Zurdo, M., Martinez-Para, M. C., 1998. Cerebrospinal fluid levels of transition metals in patients with Parkinson's disease. J Neural Transm. 105(4-5),497-505.
Lovell M. A., Robertson, J. D., Teesdale, W. J., Campbell, J. L., Markesbery, W. R., 1998. Copper, iron and zinc in Alzheimer's disease senile plaques. Journal of the neurological sciences. 158(1),47-52.
McKeith, I. G., D. W. Dickson, J. Lowe, M. Emre, J. T. O'Brien, H. Feldman, J. Cummings, J. E. Duda, C. Lippa, E. K. Perry, D. Aarsland, H. Arai, C. G. Ballard, B. Boeve, D. J. Burn, D. Costa, T. Del Ser, B. Dubois, D. Galasko, S. Gauthier, C. G. Goetz, E. Gomez-Tortosa, G. Halliday, L. A. Hansen, J. Hardy, T. Iwatsubo, R. N. Kalaria, D. Kaufer, R. A. Kenny, A. Korczyn, K. Kosaka, V. M. Lee, A. Lees, I. Litvan, E. Londos, O. L. Lopez, S. Minoshima, Y. Mizuno, J. A. Molina, E. B. Mukaetova-Ladinska, F. Pasquier, R. H. Perry, J. B. Schulz, J. Q.
Trojanowski, and M. Yamada. Diagnosis and management of dementia with Lewy bodies: third report of the DLB Consortium. Neurology. 2005. 65(12): 1863-72.
McKhann G., Drachman, D., Folstein, M., Katzman, R., Price, D., Stadlan, E. M., 1984. Clinical diagnosis of Alzheimer's disease: report of the NINCDS-ADRDA Work Group under the auspices of Department of Health and Human Services Task Force on Alzheimer's Disease. Neurology. 34(7),939-44.
Molina J. A., Jimenez-Jimenez, F. J., Aguilar, M. V., Meseguer, I., Mateos-Vega, C. J., Gonzalez-Munoz, M. J., de Bustos, F., Porta, J., Orti-Pareja, M., Zurdo, M., Barrios, E., Martinez-Para, M. C., 1998. Cerebrospinal fluid levels of transition metals in patients with Alzheimer's disease. J Neural Transm. 105(4-5),479-88.
Morgan C., Colombres, M., Nunez, M. T., Inestrosa, N. C., 2004. Structure and function of amyloid in Alzheimer's disease. Progress in neurobiology. 74(6),323-49.
Quik M, Bordia T, O'Leary K (2007). “Nicotinic receptors as CNS targets for Parkinson's disease”. Biochem. Pharmacol. 74 (8): 1224-34. doi:10.1016/j.bcp.2007.06.015. PMID 17631864.
Tamamizu-Kato S., Kosaraju, M. G., Kato, H., Raussens, V., Ruysschaert, J. M., Narayanaswami, V., 2006. Calcium-triggered membrane interaction of the α-synuclein acidic tail. Biochemistry. 45(36),10947-56.
“Effects of tocopherol and deprenyl on the progression of disability in early Parkinson's disease. The Parkinson Study Group”. N. Engl. J. Med. 328 (3): 176-83. 1993. PMID 8417384.
Todorich B. M., Connor, J. R., 2004. Redox metals in Alzheimer's disease. Ann NY Acad Sci. 1012(171-8.
Uversky V. N., Li, J., Bower, K., Fink, A. L., 2002. Synergistic effects of pesticides and metals on the fibrillation of α-synuclein: implications for Parkinson's disease. Neurotoxicology. 23(4-5),527-36.
Weisman D., McKeith, I., 2007. Dementia with Lewy bodies. Seminars in neurology. 27(1),42-7.

1. A method to determine the Mg²⁺ and/or Ca²⁺ levels in a cerebrospinal fluid obtained from a mammal, wherein Mg²⁺ and/or Ca²⁺ levels are connected to an α-synucleinopathy associated disease or disorder comprising the steps of:
a. providing a first sample of a cerebrospinal fluid obtained from a mammal
b. analysing the levels of Mg²⁺ and/or Ca²⁺ in said first sample
c. providing a second sample of a cerebrospinal fluid obtained from a mammal,
d. analysing the levels of Mg²⁺ and/or Ca²⁺ in said second sample and
e. comparing the levels of ions in said first and said second sample in order to identify differences of said ion levels in said first and said second sample.

2. The method of claim 1, wherein said first and said second samples are obtained from the same or different mammals.

3. The method according to claim 1, wherein said first sample is obtained from a mammal suffering from an α-synucleinopathy associated disease or disorder and said second sample is obtained from a healthy mammal or from a population of healthy mammals not suffering from an α-synucleinopathy associated disease or disorder.

4. The method according to claim 1, wherein said mammal is selected from the group consisting of human beings, mouse, rats, guinea pigs, cats, dogs or genetically versions thereof.

5. The method according to claim 4, wherein said mammal is human beings.

6. The method according to claim 5, wherein the human being suffering from an synucleinopathy associated disease or disorder should have a Mg²⁻ level of at least 27.3 mg/L and/or a Ca²⁺ level of at least 48.0 mg/L.

7. The method according to claim 6, wherein the human being suffering from an synucleinopathy associated disease or disorder should have a Mg² level of at least 29.3 mg/L and/or a Ca²⁺ level of at least 50.7 mg/L.

8. The method according to claim 7, wherein the human being suffering from an synucleinopathy associated disease or disorder should have a Mg²⁻ level of at least 29.3 mg/L and a Ca²⁺ level of at least 50.7 mg/L.

9. The method of claim 1, wherein the method is used to determine the progression of an α-synucleinopathy associated disease or disorder, for diagnosing an α-synucleinopathy associated disease or disorder, for tuning the dosage of a medicament against an α-synucleinopathy associated disease or disorder or for screening for pharmaceutically active compounds against α-synucleinopathy associated diseases or disorders.

10. The method according to claim 1, wherein said α-synucleinopathy associated disease or disorder is selected from the group consisting of Parkinson's disease, dementia with Lewy bodies, MSA or Hallervorden-Spatz syndrome.

11. The method according to claim 10, wherein said α-synucleinopathy associated disease or disorder is dementia with Lewy bodies.

12. A kit for the detection of Mg²⁺ and/or Ca²⁺ in cerebrospinal fluid obtained from a mammal, wherein Mg²⁺ and/or Ca²⁺ levels are connected to an α-synucleinopathy associated disease or disorder comprising a reagent for quantification of calcium and/or magnesium ions in the form of the dyes calmagite or phenolsulphonephthalein or chromatic interfering substances—vanadate ions and/or Chlorophosphanazo-III or reagents similar thereto and optionally tubes to store collected cerebrospinal fluid samples.

13. The kit according to claim 12, wherein said kit is used in a method to determine the Mg²⁺ and/or Ca²⁺ levels in a cerebrospinal fluid obtained from a mammal, wherein Mg²⁺ and/or Ca²⁺ levels are connected to an α-synucleinopathy associated disease or disorder comprising the steps of:
a. providing a first sample of a cerebrospinal fluid obtained from a mammal
b. analysing the levels of Mg²⁺ and/or Ca²⁺ in said first sample
c. providing a second sample of a cerebrospinal fluid obtained from a mammal,
d. analysing the levels of Mg²⁺ and/or Ca²⁺ in said second sample and
e. comparing the levels of ions in said first and said second sample in order to identify differences of said ion levels in said first and said second sample.

14. Use of the method according to claim 1 to identify/develop pharmaceutical compositions against α-synucleinopathy associated diseases or disorders.

15. Use according to claim 14, wherein said α-synucleinopathy associated disease or disorder is selected from the group consisting of Parkinson's disease, dementia with Lewy bodies, MSA or Hallervorden-Spatz syndrome.

16. Use according to claim 15, wherein said α-synucleinopathy associated disease or disorder is dementia with Lewy bodies.

17. Use of the method according to claim 1 to diagnose a patient with dementia.
